SEQUENCE LISTING (5 sequences)

SEQ ID NO: 1 - DNA, 2514 bp, Coccidioides posadasii
atgcagctcgtgccgcgagagatcgataaactgaccatctctaatctgggatttctggcccagcggagattggcccggggagtgaggttgaatcatgcagaagcgactgcgttgatcgcatccaatctccaagagcttattcgagatggcaacaattcggtggcagatctcatgacgatcggcaaggagatgcttggacgtcgacatgtcctaccctctgtcgtggccaccttgaaacaagttcaagttgaaggaacttttcccactgggaccaacctgatcaccgtggtcaatcccgtttgttcggatgatggcgacctggagaaagccctctacggaagctttttgcctgtgccgccgaaagaaacgttcccggatcctgatccggatgactatcagccagagaagatgcctggcgctgtgatacccctcaaaacgagcaagaaaattgagctcaatgccgggagaaatagaattatgcttaaagtgacaagtcgaggagaccggcccattcaggttggttcacactatcattttatagaggtgaatccacaattggacttcgaccgtgcgaaggcgtacggatatcgccttgacatcccagctgggacgtctatccgctttgaacccggtgctaccaaggcaatccctctcgtcgaaattggcggcaagagaatcattcgagggggcaaccacatcgccgtcgggcaagtggatttccgaagagttgatgaaatcatcatgcgtctgcaaaaagctggatttgcgtacactccggagcctaaacaggatgctcatttgatcgagccattttcaatgacgcgggaagcatatgctcgaatgtttggtcctaccactggagatgtagtcaagctaggaaccacagatttgtggattaaagtcgaaaaggacctgacctactatggtgacgaatgttcattcggtggtggcaagaccataagagacgggatggggcaagctacaggaaggcattccgtggatgtcctggatacagtcctggtgaacgcgctaattgtcgattggacgggtatttacaaggctgatattggactaaaagatggattgatctgcggaatcggcaaagctggaaacccagacatgatggatggtgtcacccccaacatgatagttggctcttcgacagatgttatcgcatgtgaaggaaaaattgtcactgcaggaggaattgacacacacgtccattttatatgcccacagcaggtcgaggaagccctcgcctccggagtcacgactcttctcggtggcggcaccggtccaactgagggatcaaatgcgactacatgcacaccggctccaaatcagttcaagacgatgatgcaggcttgtgatcatcttccaataaacgttggccttacaggcaaaggtaatgacagcggtcttccatctttgagggatcaatgccgtgcaggagccgctggcttgaaggtgcatgaagattggggtgcaacgccggctgtcattgatacacgcctccaggtctgcgacgagttcgatattcaatgtctcatccataccgacaccctgaacgaatctggcttcgttgaacagaccatcaatgcctttaaaaatcgagtgattcatacgtaccacactgagggtgctggaggaggccacgctccagatatcatatccgtcgtcgagaagccaaacgtcctgcccagcagtacgaatcccactcgtccgtatacggtaaatactttagatgaacatctggacatggtaatggtctgccatcatttgtccaaagatattcctgaagacgtggcttttgcggaaagccggatccgatccgagacaattgctgcagaagacgttcttcatgacacgggagccatcagcatgctatcctcggactctcaagctatgggacgctgtggagaagttgttgttcggacatggaacactgcacataagaataaaacggaacgagggcgactcaaggaagatgaagggacggattctgataattttagggttaaacggtatatcagcaagtacaccatcaaccctgccattgcacaggggatggcccacactattgggagcgtggaagttggcaagaccgctgatttggttctgtggaaatttgccaaccttgggactaaaccgagtatggtcttgaagtctggaatggctgtctcagcgcaggtgggtgatcccaatggctctatccccacaatcgagcctattattatgaggcctatgtacgctagtcttaaccctaaagcctcaatcatgttcgtatcccaagcatccatcaagcttggtatcatcgacagttaccacctgaagaagcggatcgagccagtgaagaattgtcggaatataagcaagagagatatgaaatttaatgatattatgcccaaaatgagagtcgatccggagagctatgttgtcgaggctgacggggaagagtgcaccgctgagccagtgtcagagttgcctttaacacaagattatttcgtttactga

SEQ ID NO: 2 - protein, 837 aa, Coccidioides posadasii
MQLVPREIDKLTISNLGFLAQRRLARGVRLNHAEATALIASNLQELIRDGNNSVADLMTIGKEMLGRRHVLPSVVATLKQVQVEGTFPTGTNLITVVNPVCSDDGDLEKALYGSFLPVPPKETFPDPDPDDYQPEKMPGAVIPLKTSKKIELNAGRNRIMLKVTSRGDRPIQVGSHYHFIEVNPQLDFDRAKAYGYRLDIPAGTSIRFEPGATKAIPLVEIGGKRIIRGGNHIAVGQVDFRRVDEIIMRLQKAGFAYTPEPKQDAHLIEPFSMTREAYARMFGPTTGDVVKLGTTDLWIKVEKDLTYYGDECSFGGGKTIRDGMGQATGRHSVDVLDTVLVNALIVDWTGIYKADIGLKDGLICGIGKAGNPDMMDGVTPNMIVGSSTDVIACEGKIVTAGGIDTHVHFICPQQVEEALASGVTTLLGGGTGPTEGSNATTCTPAPNQFKTMMQACDHLPINVGLTGKGNDSGLPSLRDQCRAGAAGLKVHEDWGATPAVIDTRLQVCDEFDIQCLIHTDTLNESGFVEQTINAFKNRVIHTYHTEGAGGGHAPDIISVVEKPNVLPSSTNPTRPYTVNTLDEHLDMVMVCHHLSKDIPEDVAFAESRIRSETIAAEDVLHDTGAISMLSSDSQAMGRCGEVVVRTWNTAHKNKTERGRLKEDEGTDSDNFRVKRYISKYTINPAIAQGMAHTIGSVEVGKTADLVLWKFANLGTKPSMVLKSGMAVSAQVGDPNGSIPTIEPIIMRPMYASLNPKASIMFVSQASIKLGIIDSYHLKKRIEPVKNCRNISKRDMKFNDIMPKMRVDPESYVVEADGEECTAEPVSELPLTQDYFVY

SEQ ID NO: 3 - DNA, 1749 bp, Coccidioides posadasii
atgggcagcagccatcatcatcatcatcacagcagcggcctggtgccgcgcggcagccatatggctagcatgactggtggacagcaaatgggtcgggatccacaattggacttcgaccgtgcgaaggcgtacggatatcgccttgacatcccagctgggacgtctatccgctttgaacccggtgctaccaaggcaatccctctcgtcgaaattggcggcaagagaatcattcgagggggcaaccacatcgccgtcgggcaagtggatttccgaagagttgatgaaatcatcatgcgtctgcaaaaagctggatttgcgtacactccggagcctaaacaggatgctcatttgatcgagccattttcaatgacgcgggaagcatatgctcgaatgtttggtcctaccactggagatgtagtcaagctaggaaccacagatttgtggattaaagtcgaaaaggacctgacctactatggtgacgaatgttcattcggtggtggcaagaccataagagacgggatggggcaagctacaggaaggcattccgtggatgtcctggatacagtcctggtgaacgcgctaattgtcgattggacgggtatttacaaggctgatattggactaaaagatggattgatctgcggaatcggcaaagctggaaacccagacatgatggatggtgtcacccccaacatgatagttggctcttcgacagatgttatcgcatgtgaaggaaaaattgtcactgcaggaggaattgacacacacgtccattttatatgcccacagcaggtcgaggaagccctcgcctccggagtcacgactcttctcggtggcggcaccggtccaactgagggatcaaatgcgactacatgcacaccggctccaaatcagttcaagacgatgatgcaggcttgtgatcatcttccaataaacgttggccttacaggcaaaggtaatgacagcggtcttccatctttgagggatcaatgccgtgcaggagccgctggcttgaaggtgcatgaagattggggtgcaacgccggctgtcattgatacacgcctccaggtctgcgacgagttcgatattcaatgtctcatccataccgacaccctgaacgaatctggcttcgttgaacagaccatcaatgcctttaaaaatcgagtgattcatacgtaccacactgagggtgctggaggaggccacgctccagatatcatatccgtcgtcgagaagccaaacgtcctgcccagcagtacgaatcccactcgtccgtatacggtaaatactttagatgaacatctggacatggtaatggtctgccatcatttgtccaaagatattcctgaagacgtggcttttgcggaaagccggatccgatccgagacaattgctgcagaagacgttcttcatgacacgggagccatcagcatgctatcctcggactctcaagctatgggacgctgtggagaagttgttgttcggacatggaacactgcacataagaataaaacggaacgagggcgactcaaggaagatgaagggacggattctgataattttagggttaaacggtatatcagcaagtacaccatcaaccctgccattgcacaggggatggcccacactattgggagcgtggaagttggcaagaccgctgatttggttctgtggaaatttgccaaccttgggactaaaccgagtatggtcttgaagtctggaatggctgtctcagcgcaggtggtatga

SEQ ID NO: 4 - protein, 582 aa, coccidioides posadasii
MGSSHHHHHHSSGLVPRGSHMASMTGGQQMGRDPQLDFDRAKAYGYRLDIPAGTSIRFEPGATKAIPLVEIGGKRIIRGGNHIAVGQVDFRRVDEIIMRLQKAGFAYTPEPKQDAHLIEPFSMTREAYARMFGPTTGDVVKLGTTDLWIKVEKDLTYYGDECSFGGGKTIRDGMGQATGRHSVDVLDTVLVNALIVDWTGIYKADIGLKDGLICGIGKAGNPDMMDGVTPNMIVGSSTDVIACEGKIVTAGGIDTHVHFICPQQVEEALASGVTTLLGGGTGPTEGSNATTCTPAPNQFKTMMQACDHLPINVGLTGKGNDSGLPSLRDQCRAGAAGLKVHEDWGATPAVIDTRLQVCDEFDIQCLIHTDTLNESGFVEQTINAFKNRVIHTYHTEGAGGGHAPDIISVVEKPNVLPSSTNPTRPYTVNTLDEHLDMVMVCHHLSKDIPEDVAFAESRIRSETIAAEDVLHDTGAISMLSSDSQAMGRCGEVVVRTWNTAHKNKTERGRLKEDEGTDSDNFRVKRYISKYTINPAIAQGMAHTIGSVEVGKTADLVLWKFANLGTKPSMVLKSGMAVSAQVV

SEQ ID NO: 5 - DNA, 20 bp, Synthesized
tccatgacgttcctgacgtt

(19) United States
(12) Patent Application Publication
(10) Pub. No.: US 2003/0224013 A1
(43) Pub. Date: Dec. 4, 2003

(54) Methods for protection against Coccidioides spp. infection using Coccidioides spp. urea amidohydrolase (Ure) protein

(76) Inventors: Garry T. Cole, Toledo, OH (US); Jieh-Juen Yu, Toledo, OH (US); Kun Li, Ann Arbor, MI (US); Kalpathi Seshan, Toledo, OH (US)

Correspondence Address: Catherine B. Martineau, Emch, Schaffer, Schaub & Porcello Co., L.P.A., P.O. Box 916, Toledo, OH, 43697-0916, US

(21) Appl. No.: 10/418,962

(22) Filed: Apr. 18, 2003

Related U.S. Application Data
(60) Provisional application No. 60/373,634, filed on Apr. 19, 2002.

(51) Int. Cl. A61K 39/005; A61K 39/008; C07H 21/04; C12N 1/10; C12N 15/74; C07K 14/44

(52) U.S. Cl. 424/191.1; 424/269.1; 435/69.3; 435/258.1; 435/320.1; 530/350; 536/23.6

(57) ABSTRACT

The present invention provides compositions and methods of use of urea amidohydrolase (Ure) antigens and polynucleotides encoding the Ure antigens for generating an immunological response in an individual and in therapeutic and diagnostic applications of infections due to pathogenic Coccidioides spp. fungi, such as C. immitis or C. posadasii.

### RELATED APPLICATIONS

This application claims benefit of priority under 35 U.S.C. 119(B) of Provisional Application Serial No. 60/373,634 filed Apr. 19, 2002, the contents of which are incorporated herein by reference.

### STATEMENT REGARDING FEDERALLY FUNDED PROJECT

The United States Government has a paid-up license in this invention and the right in limited circumstances to require the patent owner to license others on reasonable terms as provided for by the terms of Public Service Grants AI19149 and AI37232.

### FIELD OF THE INVENTION

The present invention relates generally to the fields of pathogenic fungi and immunology. In particular, this invention provides compositions and methods of use of Coccidioides spp. polypeptide urea amidohydrolase (Ure) antigens and polynucleotides encoding the Ure antigens for generating or detecting an immunological response and in vaccines and therapeutic applications for infections due to pathogenic Coccidioides spp. fungi, such as C. posadasii or C. immitis.

### BACKGROUND OF THE INVENTION

Coccidioidomycosis, otherwise known as the San Joaquin Valley Fever, is a fungal respiratory disease of humans and wild and domestic animals which is endemic to southwestern United States, northern Mexico, and numerous semiarid areas of Central and South America (Pappagianis, D. Epidemiology of Coccidioidomycosis. Current Topics in Medical Mycology. 1988. 2:199-23). Infection occurs by inhalation of airborne spores (arthroconidia) produced by the saprobic phase of Coccidioides spp. which grows in alkaline desert soil. C. immitis was the first described species, and is now becoming known as the Californian species. The C. posadasii species was recently defined, and was previously recognized as the non-Californian population of C. immitis (Fisher, M. C., Koenig, G. L., White, T. J., Taylor, J. W. Molecular and phenotypic description of Coccidioides posadasii sp. nov., previously recognized as the non-California population of Coccidioides immitis. Mycologia 2002. 94(1):73-84, 2002). The differences in the two species are slight.

It is estimated that 100,000 new cases of this disease occur annually within the rapidly growing population of people who live in regions of the United States between southwest Texas and southern California, where the disease is endemic (Galgiani, J. N. Coccidioidomycosis: A regional disease of national importance; rethinking our approaches to its control. Annals of Internal Medicine. 1999. 130:293-300). Although the majority of immunocompetent individuals are able to resolve their Coccidioides spp. infection spontaneously, the level of morbidity associated even with the primary form of this respiratory mycosis warrants consideration of a vaccine against the disease. Immunocompromised patients, including those infected with human immunodeficiency virus, are at high risk to contract disseminated coccidioidomycosis (Ampel, N. M., C. L. Dols, and J. N. Galgiani. Results of a prospective study in a coccidioidal endemic area. American Journal of Medicine. 1993. 94:235-240). It is also apparent from results of several clinical studies that African-Americans and Asians are genetically predisposed to development of the potentially fatal, disseminated form of the respiratory disease (Galgiani, J. N. 1993. Coccidioidomycosis. Western Journal of Medicine 159:153-171).

The rationale for commitment of research efforts to develop a Coccidioides spp. vaccine is based on clinical evidence that individuals who recover from the respiratory coccidioidomycosis disease retain effective long-term cellular immunity against future infections by the pathogen (Smith, C. E. 1940. American Journal of Public Health 30:600-611). In addition, early preclinical studies demonstrated that a formalin-killed whole-cell (spherule) vaccine prevented deaths in mice after infection with even very large numbers of coccidioidal spores (Levine et al. 1961. Journal of Immunology 87:218-227). However, when a similar vaccine preparation was evaluated in a human trial, there was substantial local inflammation, pain, and induration at the injection site, rendering the vaccine unacceptable (Pappagianis et al. Evaluation of the protective efficacy of the killed Coccidioides immitis spherule vaccine in humans. American Review of Respiratory Diseases. 1993. 148:656-660). Further, there was no difference in the number of cases of coccidioidomycosis or the severity of the disease in the formalin-killed spherule vaccinated group compared to the placebo group. Therefore, the original human vaccine trial was not successful.

Subsequent attempts to develop a coccidioidal vaccine focused on crude or partially purified subcellular preparations from the fungus, and had limited success in experimental models (Zimmermann, C. R., S. M. Johnson, G. W. Martens, A. G. White, B. L. Zimmer, and D. Pappagianis. Protection against lethal murine coccidioidomycosis by a soluble vaccine from spherules. Infection and Immunity. 1988. 66:2342-2345; Lecara, G., Cox, R. A., and Simpson, R. B. Coccidioides immitis vaccine: potential of an alkali-soluble, water-soluble cell wall antigen. Infection and Immunity. 1983. 39: 473-475; Cole, G. T., T. N. Kirkland, and S. H. Sun. An immunoreactive, water-soluble conidial wall fraction of Coccidioides immitis 1987. Infection and Immunity 55:657-667; Cole G. T., Kirkland T. N., Franco M., Zhu S., Yuan L., Sun S. H., Hearn V. M. Immunoreactivity of a surface wall fraction produced by spherules of Coccidioides immitis. Infection and Immunity October 1988; 56:2695-701).

Another approach has been to use mechanical disruption to obtain Coccidioides spp. antigens (Cole, G. T., T. N. Kirkland, and S. H. Sun. 1987. An immunoreactive, water-soluble conidial wall fraction of Coccidioides immitis. Infection and Immunity 55:657-667). The advantages of this method are that it avoids chemical extraction and autolysins, which may alter the native structure of antigens, and it yields reproducible preparations of intact proteins. Upon removing the outer conidial wall from arthroconidia via this approach, the organism releases a variety of proteins in a soluble conidial wall fraction. This mixture of proteins has been shown to be extraordinarily effective in stimulating immune murine T-cell proliferation but not effective as a vaccine against coccidioidomycosis. This mixture also contains proteolytic antigens, which could have a negative impact on the host response.

The spherule phase of the organism spontaneously releases a membranous material consisting primarily of glycosylated proteins and lipids known as the spherule outer wall, or SOW (Cole G T, Kirkland T N, Franco M, Zhu S, Yuan L, Sun S H, Hearn V M. 1988. Infection and Immunity 56:2695-2701). This wall fraction has been shown to be a potent antigen in T-cell mediated immune responses, both in mice and humans, however, this fraction did not, under the experimental conditions employed at that time, provide significant protection to mice against infection with Coccidioides immitis.

In a search for virulence factors of Coccidioides spp, a urease gene (URE) has been identified (Yu, J. -J., Smithson, S. L., Thomas, P. W., Kirkland, T. N., and Cole, G. T. 1997. Isolation and characterization of the urease gene (URE) from the pathogenic fungus Coccidioides immitis, Gene 198:387-391). The coccidioidal urease gene has been shown to be expressed in vivo, and there are suggestions that it plays a role in both sporulation and pathogenesis (Cole, G. T. 1997. Ammonia production by Coccidioides immitis and its possible significance to the host-fungus interplay, p. 247-263. In H. van den Bossche, D. A. Stevens, and F. C. Odds (ed.), Host-fungus interplay. National Foundation for Infectious Diseases, Bethesda, Md.).

Unfortunately, although certain immunization strategies have been explored, there remains in the art a need to identify biological components that generate effective immune responses against Coccidioides spp. The identification of smaller immunogenic components and/or those that elicit cell-mediated immune responses suitable for treatment or as a vaccination regimen to prevent infection of Coccidioides spp. and disease states associated with the infection would be a particular advance in this area.

### SUMMARY OF THE INVENTION

The present invention overcomes various drawbacks and addresses the long felt need in the art by providing Coccidioides spp. polypeptide compositions for prophylactic and therapeutic uses. It is well known in the art that DNA vaccines have potential safety issues, including induction of autoimmunity and insertional mutagenesis in the recipient host, and represent other unique challenges for regulatory approval for use in humans and domestic animals. Protein and peptides, in contrast, and known to be strong inducers of MCH II type responses, and such responses are known to be of importance in the acquisition of immunity to infection by Coccidioides spp.

Accordingly, it is an object herein to provide polypeptides and polynucleotides encoding polypeptides of Coccidioides spp. that have an immunostimulatory activity. Such immunostimulatory polypeptides will be useful in the prevention, treatment, and diagnosis of infections due to Coccidioides spp.

In order to meet these needs, the present invention provides compositions and methods of use of Coccidioides spp. urea amidohydrolase (Ure) polypeptides and polynucleotides encoding the polypeptides to elicit an immune response sufficient to provide an effective immunization against Coccidioides spp. infection, including but not limited to the polypeptide sequences of SEQ ID NO:2 and or SEQ ID NO:4. In one embodiment, the polypeptides provide protection against Coccidioides posadasii and or Coccidioides immitis infections in a mammal, such as a human. In another embodiment, the polypeptides provide protection against Coccidioides spp. infection in domestic animals, including but not limited to dogs, cats, horses, and cattle. In an additional embodiment, the polypeptides of the present invention encompasses the full-length 582 amino acid recombinant Ure fusion protein sequence of SEQ ID NO:4. In another embodiment, the present invention comprises the full length recombinant polypeptide sequence lacking the N-terminal fusion peptide amino acids 1 to 33 of SEQ ID NO:4.

The present invention further provides the compositions and the methods of use of the Ure polypeptides in combination with one or more other Coccidioides spp. antigens to elicit an immune response sufficient to provide an effective immunization against Coccidioides spp. infection. In one embodiment the polypeptides are provided as a composition containing a mixture of said polypeptides, for example, Ure in combination with Ag2/PRA1-106 (Peng T, Shubitz L, Simons J, Perrill R, Orsborn K I, Galgiani J N. 2002. Localization within Antigen 2/PRA of protective antigenicity for mice against infection with Coccidioides immitis. Infection and Immunity 70(7):3330-3335) and or Coccidioides-immitis specific antigen (referred to hereafter as Csa) (Pan, S. and Cole, G. T. 1995. Molecular and biochemical characterization of Coccidioides immitis-specific (CS) antigen. Infection and Immunity, 63:3994-4002). In another embodiment the composition is provided as a single fusion polypeptide comprised of the Coccidioides spp. polypeptides.

The present invention also provides vaccine formulations and methods of preparing the formulations containing the Ure polypeptides for use in eliciting an immune response in mammals for the prevention of Coccidioides spp. infections. The present invention further provides vaccine formulations containing the polypeptides, adjuvants and pharmaceutical excipients and carriers.

The present invention further provides kits containing the Ure polypeptides and or other Coccidioides spp. antigens and or polynucleotides encoding the polypeptides, to facilitate the use of the polypeptides and or polynucleotides as immunizing vaccines.

The above and other aspects of the invention will become readily apparent to those of skill in the art from the following detailed description and figures, wherein only the preferred embodiments of the invention are shown and described, simply by way of illustration of the best mode of carrying out the invention. As is readily recognized, the invention is capable of modifications within the skill of the relevant art without departing from the spirit and scope of the invention.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. The aligned nucleotide (SEQ ID NO:3) and deduced amino acid sequences (SEQ ID NO:4) of the rURE gene construct expressed in an E. coli host. The construct includes 99 nucleotides encoding a 33 amino acid fusion peptide on the N-terminal derived from the pET28b vector. The translated amino acid sequence shows a protein of 582 residues, which includes the vector-encoded fusion partner peptide.

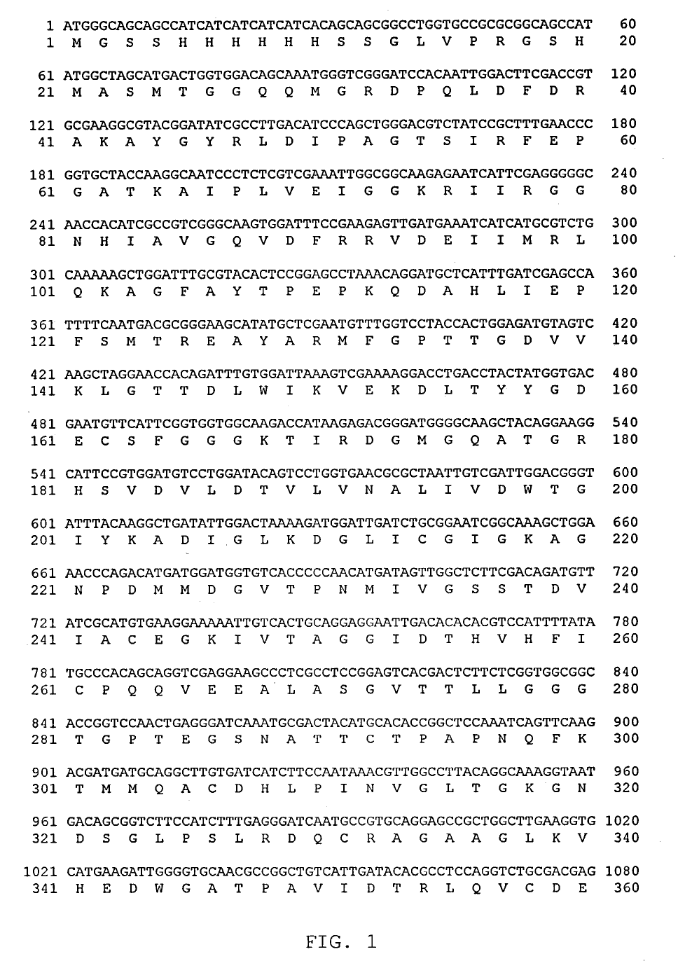

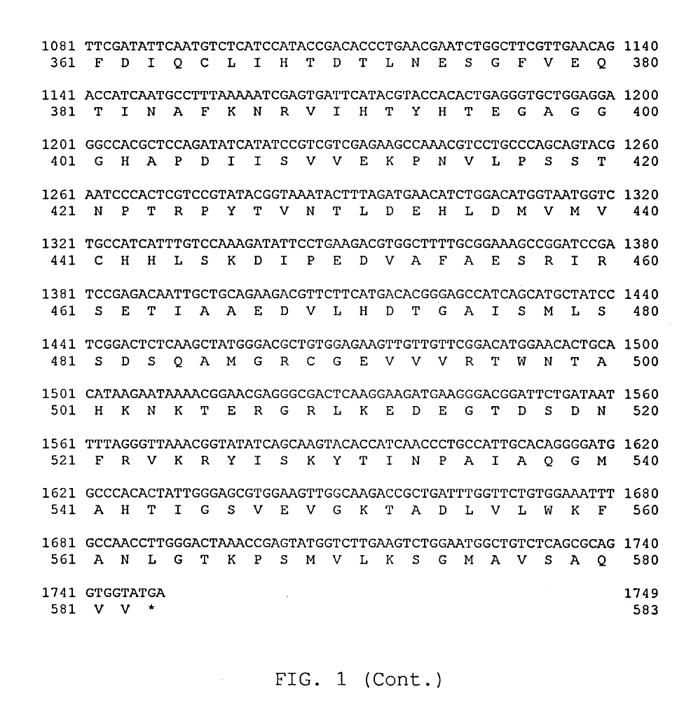

### BRIEF DESCRIPTION OF THE SEQUENCE LISTING

SEQ ID NO:1 depicts the determined cDNA nucleotide sequence encoding the full-length native Ure polypeptide;

SEQ ID NO:2 depicts the deduced amino acid sequence of the native Ure polypeptide encoded by the nucleotide sequence of SEQ ID NO:1;

SEQ ID NO:3 depicts the determined nucleotide sequence of the pET28b recombinant construct encoding the recombinant Ure fusion polypeptide;

SEQ ID NO:4 depicts the deduced amino acid sequence of the recombinant Ure fusion polypeptide encoded by the nucleotide sequence of SEQ ID NO:3, including 33 N-terminal amino acids derived from the pET28b vector;

SEQ ID NO:5 depicts the nucleotide sequence of the synthetic CpG adjuvant used in animal experiments.

### DETAILED DESCRIPTION OF THE INVENTION

Although methods and materials similar or equivalent to those described herein can be used in the practice or testing of the present invention, suitable methods and materials are described herein. All publications, patent applications, patents, and other references mentioned herein are incorporated by reference in their entirety. In addition, the materials, methods, and examples are illustrative only and are not intended to be limiting.

Reference is made to standard textbooks of molecular biology that contain definitions and methods and means for carrying out basic techniques, encompassed by the present invention. See, for example, Sambrook et al., Molecular Cloning: A Laboratory Manual, Third Edition, Cold Spring Harbor Laboratory Press, New York (2001), Current Protocols in Molecular Biology, Ausubel et al (eds.), John Wiley & Sons, New York (2001) and the various references cited therein.

I. The Polypeptide Sequences of the Invention.

The invention focuses on methods of use of Ure polypeptides and the nucleotide sequences that encode them as immunogenic antigens for a preventative or therapeutic vaccine for coccidioidomycosis, or for detection of immune responses in individuals infected by Coccidioides spp.

Native Ure from Coccidioides spp. was originally described as a protein of 839 amino acids, deduced from the original DNA sequence data reported by Yu et al. (Yu, J. -J., Smithson, S. L., Thomas, P. W., Kirkland, T. N., and Cole, G. T. 1997. Isolation and characterization of the urease gene (URE) from the pathogenic fungus Coccidioides immitis. Gene 198:387-391). Subsequently, resequencing efforts determined that there were substantive errors in the published DNA sequences and that the native protein consists of 837 amino acids, with 40 residues that differed from the original sequence. The revised amino acid sequence of native Ure is shown in SEQ ID NO:2. Similarly, the recombinant Ure, originally reported as containing 584 amino acid residues, was revised to 582 amino acids with 33 specific amino acid differences from the original sequence. The rUre is a truncated version of the native protein, consisting of 582 amino acids and includes a 33 amino acid fusion partner peptide at the N-terminal of the rUre polypeptide (MGSSHHHHHHSSGLVPRGSHMASMTGGQQMGRD) derived from the pET-28b vector. The revised amino acid sequence of rUre is shown in SEQ ID NO:4.

Use of additional polypeptides as immunizing antigens are encompassed in the invention, consisting essentially of the sequences of the Ure and or rUre polypeptides, including polypeptides at least about 99% identical or equivalent, at least about 95% identical or equivalent, at least about 90% identical or equivalent, at least about 85% identical or equivalent, at least about 80% identical or equivalent, at least about 75% identical or equivalent, and at least about 70% identical to the sequences of the polypeptides.

As used herein, the terms “protein” or “polypeptide” are used in the broadest sense to mean a sequence of amino acids that can be encoded by a cellular gene or by a recombinant nucleic acid sequence or can be chemically synthesized. A protein can be a complete, full length gene product, which can be a core protein having no amino acid modifications, or can be a post-translationally modified form of a protein such as a phosphoprotein, glycoprotein, proteoglycan, lipoprotein or nucleoprotein. In some cases, the term “polypeptide” is used in referring to a portion of an amino acid sequence (peptides) of a full length protein. An active fragment of a Ure is an example of such a polypeptide. The reasons for reducing the full-length antigen to fragments are multiple; 1) a positive correlation between development of DTH (delayed-type hypersensitivity) (a Th1 immune response) to coccidioidal antigens and the ability to resist disseminated coccidioidomycosis has been shown. This response is generally regarded as a MHC II type response (Louie et al. 1999. Influence of host genetics on the severity of coccidioidomycosis. Emerging Infectious Diseases 5:672-680) and that peptides that bind to MHC II receptors are generally 13-25 residues in length; 2) determination of epitopes within a larger polypeptide or multiple peptides that enhance or suppress an immune response allow for the creation of fusion proteins containing select epitopes that can be used as vaccines with increased potency (Sette A, et al. 2002. Optimizing vaccine design for cellular processing, MHC binding and TCR recognition. Tissue Antigens 59:443-451); and 3) reducing the size of the polypeptide can lead to important advantages in the production, purification and safety of a vaccine.

“Consisting essentially of”, in relation to amino acid sequence of a polypeptide, protein or peptide, is a term used hereinafter for the purposes of the specification and claims to refer to a conservative substitution or modification of one or more amino acids in that sequence such that the tertiary configuration of the polypeptide, protein or peptide is substantially unchanged. Polypeptides, consisting essentially of the sequence of the polypeptides of Ure and or rUre, include those polypeptides at least about 99% identical or equivalent, at least about 95% identical or equivalent, at least about 90% identical or equivalent, at least about 85% identical or equivalent, at least about 80% identical or equivalent, at least about 75% identical or equivalent, and at least about 70% identical to the sequences of the polypeptides.

“Conservative substitutions” is defined by substitutions of amino acids having substantially the same charge, size, hydrophilicity, and or aromaticity as the amino acid replaced. Such substitutions, known to those of ordinary skill in the art, include glycine-alanine-valine; isoleucine-leucine; tryptophan-tyrosine; aspartic acid-glutamic acid; arginine-lysine; asparagine-glutamine; and serine-threonine.

“Modification”, in relation to amino acid sequence of a polypeptide, protein or peptide, is defined functionally as a deletion of one or more amino acids which does not impart a change in the conformation, and hence the biological activity, of the polypeptide, protein or peptide sequence.

The common amino acids are generally known in the art. Additional amino acids that may be included and or substituted in the peptide of the present invention include: L-norleucine; aminobutyric acid; L-homophenylalanine; L-norvaline; D-alanine; D-cysteine; D-aspartic acid; D-glutamic acid; D-phenylalanine; D-histidine; D-isoleucine; D-lysine; D-leucine; D-methionine; D-asparagine; D-proline; D-glutamine; D-arginine; D-serine; D-threonine; D-valine; D-tryptophan; D-tyrosine; D-omithine; aminoisobutyric acid; L-ethylglycine; L-t-butylglycine; penicillamine; I-naphthylalanine; cyclohexylalanine; cyclopentylalanine; aminocyclopropane carboxylate; aminonorbornylcarboxylate; L-α-methylalanine; L-α-methylcysteine; L-α-methylaspartic acid; L-α-methylglutamic acid; L-α-methylphenylalanine; L α-methylhistidine; L-α-methylisoleucine; L-α-methyllysine; L-α-methylleucine; L-α-methylmethionine; L-α-methylasparagine; L-α-methylproline; L-α-methylglutamine; L-α-methylarginine; L-α-methylserine; L-α-methylthreonine; L-a-methylvaline; L-α-methyltryptophan; L-α-methyltyrosine; L-α-methylornithine; L-α-methylnorleucine; amino-α-methylbutyric acid;. L-α-methylnorvaiine; L-α-methylhomophenylalanine; L-α-methylethylglycine; methyl-γ-aminobutyric acid; methylaminoisobutyric acid; L-α-methyl-t-butylglycine; methylpenicillamine; methyl-α-naphthylalanine; methylcyclohexylalanine; methylcyclopentylalanine; D-α-methylalanine; D-α-methylornithine; D-α-methylcysteine; D-α-methylaspartic acid; D-α-methylglutamic acid; D-α-methylphenylalanine; D-α-methylhistidine; D-α-methylisoleucine; D-α-methyllysine; D-α-methylleucine; D-α-methylmethionine; D-α-methylasparagine; D-α-methylproline; D-α-methylglutamnine; D-α-methylarginine; D-α-methylserine; D-α-methylthreonine; D-α-methylvaline; D-α-methyltryptophan; D-α-methyltyrosine; L-N-methylalanine; L-N-methylcysteine; L-N-methylaspartic acid; L-N-methylglutamic acid; L-N-methylphenylalanine; L-N-methylhistidine; L-N-methylisoleucine; L-N-methyllysine; L-N-methylleucine; L-N-methylmethionine; L-N-methylasparagine; N-methylcyclohexylalanine; L-N-methylglutamine; L-N-methylarginine; L-N-methylserine; L-N-methylthreonine; L-N-methylvaline; L-N-methyltryptophan; L-N-methyltyrosine; L-N-methylomithine; L-N-methylnorleucine; N-amino-α-methylbutyric acid; L-N-methylnorvaline; L-N-methylhomophenylalanine; L-N-methylethylglycine; N-methyl-γaminobutyric acid; N-methylcyclopentylalanine; L-N-methyl-t-butylglycine; N-methylpenicillamine; N-methyl-α-naphthylalanine; N-methylaminoisobutyric acid; N-(2-aminoethyl)glycine; D-N-methylalanine; D-N-methylomithine; D-N-methylcysteine; D-N-methylaspartic acid; D-N-methylglutamic acid; D-N-methylphenylalanine; D-N-methylhistidine; D-N-methylisoleucine; D-N-methyllysine; D-N-methylleucine; D-N-methylmethionine; D-N-methylasparagine; D-N-methylproline; D-N-methylglutamine; D-N-methylarginine; D-N-methylserine; D-N-methylthreonine; D-N-methylvaline; D-N-methyltryptophan; D-N-methyltyrosine; N-methylglycine; N-(carboxymethyl)glycine; N-(2-carboxyethyl)glycine; N-benzylglycine; N-(imidazolylethyl)glycine; N-(1-methylpropyl)glycine; N-(4-aminobutyl)glycine; N-(2-methylpropyl)glycine; N-(2-methylthioethyl)glycine; N-(hydroxyethyl)glycine; N-(carbamylmethyl)glycine; N-(2-carbamylethyl)glycine; N-(1-methylethyl)glycine; N-(3-guanidinopropyl)glycine; N-(3-indolylethyl)glycine; N-(p-hydroxyphenethyl)glycine; N-(1-hydroxyethyl)glycine; N-(thiomethyl)glycine; N-(3-aminopropyl)glycine; N-cyclopropylglycine; N-cyclobutyglycine; N-cyclohexylglycine; N-cycloheptylglycine; N-cyclooctylglycine; N-cyclodecylglycine; N-cycloundecylglycine; N-cyclododecylglycine; N-(2,2-diphenylethyl)glycine; N-(3,3-diphenylpropyl)glycine; N-(N-(2,2-diphenylethyl)carbamylmethyl)glycine; N-(N-(3,3-diphenylpropyl)carbamylmethyl)glycine; and 1-carboxy-1-(2,2-diphenylethylamino)cyclopropane.

The polypeptides of the present invention can be produced by a known chemical synthesis method based on that sequence; for example, a liquid phase synthesis method, a solid phase synthesis method, and others (Izumiya, N., Kato, T., Aoyagi, H., Waki, M., “Basis and Experiments of Peptide Synthesis”, 1985, Maruzen Co., Ltd.).

The polypeptides of the present invention may contain one or more protected amino acid residues. The protected amino acid is an amino acid whose functional group or groups is/are protected with a protecting group or groups by a known method or by the use of various protected amino acids that are commercially available.

II. The DNA Sequences of the Invention.

Alternatively, the polypeptides antigens of the present invention can be produced by producing a polynucleotide (DNA or RNA) which encodes the amino acid sequence of a polypeptide of the present invention and producing said polypeptide by a genetic engineering technique using the polynucleotide. Polynucleotide coding sequences for amino acid residues are known in the art and are disclosed for example in Molecular Cloning: A Laboratory Manual, Third Edition, Sambrook, Fritsch, and Maniatis, Cold Spring Harbor Laboratory Press, 2001.

The cDNA URE sequence that encodes the native Ure polypeptide was originally reported by Yu et al. (Yu, J. -J., Smithson, S. L., Thomas, P. W., Kirkland, T. N., and Cole, G. T. 1997. Isolation and characterization of the urease gene (URE) from the pathogenic fungus Coccidioides immitis. Gene 198:387-391), as consisting of 2520 nucleotides, but was subsequently found through resequencing to have substantive errors. The revised cDNA sequence encoding native Ure contains 2514 nucleotides, with differences in 24 specific nucleotides is shown in SEQ ID NO:1. The nucleotide sequence of the recombinant URE construct, also revised from 1755 nucleotides to 1749 nucleotides after resequencing, had differences in 21 specific nucleotides. The rURE construct is shown in SEQ ID NO:3, which includes 99 nucleotides derived from the pET28b vector.

Within the context of the present invention “polynucleotide” in general relates to polyribonucleotides and polydeoxyribonucleotides, it being possible for these to be non-modified RNA or DNA or modified RNA or DNA. Polynucleotides which encode the peptides of the present invention mean the sequences exemplified in this application as well as those which have substantial identity to those sequences and which encode the peptides. Preferably, such polynucleotides are those which hybridize under stringent conditions as defined herein and are at least 70%, preferably at least 80% and more preferably at least 90% to 95% identical to those sequences.

“Consisting essentially of”, in relation to a nucleic acid sequence, is a term used hereinafter for the purposes of the specification and claims to refer to sequences of the present invention and sequences with substitution of nucleotides as related to third base degeneracy. As appreciated by those skilled in the art, because of third base degeneracy, almost every amino acid can be represented by more than one triplet codon in a coding nucleotide sequence. Further, minor base pair changes may result in variation (conservative substitution) in the amino acid sequence encoded, and are not expected to substantially alter the biological activity of the gene product. Thus, a nucleic acid sequencing encoding a protein or peptide as disclosed herein, may be modified slightly in sequence (e.g., substitution of a nucleotide in a triplet codon), and yet still encode its respective gene product of the same amino acid sequence.

The terms “stringent conditions” or “stringent hybridization conditions” includes reference to conditions under which a polynucleotide will hybridize to its target sequence, to a detectably greater degree than other sequences (e.g., at least 2-fold over background). In particular, a DNA or polynucleotide molecule which hybridizes under stringent conditions is preferably at least 70%, 75%, 80%, 85%, 90%, 95%, 97%, 98% or 99% identical to the DNA that encodes the amino acid sequences described herein. In a preferred embodiment these polynucleotides that hybridize under stringent conditions also encode a protein or peptide which upon administration to a subject provides an immunostimulation sufficient to provide some level of immune protection against Coccidioides spp. infection as described herein.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short polynucleotides (e.g., 10 to 50 nucleotides) and at least about 60° C. for long polynucleotides (e.g., greater than 50 nucleotides)—for example, “stringent conditions” can include hybridization in 50% formamide, 1 M NaCl, 1% SDS at 37° C., and three washes, 15 minutes each, in 0.1× SSC at 60 to 65° C.

Homology, sequence similarity or sequence identity of nucleotide or amino acid sequences may be determined conventionally by using known software or computer programs such as the BestFit or Gap pairwise comparison programs (GCG Wisconsin Package, Genetics Computer Group, 575 Science Drive, Madison, Wis. 53711). BestFit uses the local homology algorithm of Smith and Waterman (Advances in Applied Mathematics. 1981. 2: 482-489), to find the best segment of identity or similarity between two sequences. Gap performs global alignments: all of one sequence with all of another similar sequence using the method of Needleman and Wunsch, (Journal of Molecular Biology. 1970. 48:443-453). When using a sequence alignment program such as BestFit to determine the degree of sequence homology, similarity or identity, the default setting may be used, or an appropriate scoring matrix may be selected to optimize identity, similarity or homology scores. Similarly, when using a program such as BestFit to determine sequence identity, similarity or homology between two different amino acid sequences, the default settings may be used, or an appropriate scoring matrix, such as blosum45 or blosum80, may be selected to optimize identity, similarity or homology scores.

Naturally, the present invention also encompasses DNA segments that consist essentially of or are complementary, or essentially complementary, to the sequence set forth in SEQ ID NO:1 and SEQ ID NO:3. Nucleic acid sequences that are “complementary” are those that are capable of base-pairing according to the standard Watson-Crick complementarity rules. As used herein, the term “complementary sequences” means nucleic acid sequences that are substantially complementary, as may be assessed by the same nucleotide comparison set forth above, or as defined as being capable of hybridizing to the nucleic acid segment SEQ ID NO:1 or SEQ ID NO:3 under stringent conditions such as those described herein.

The nucleic acid segments of the present invention, regardless of the length of the coding sequence itself, may be combined with other nucleic acid and DNA sequences, such as promoters, polyadenylation signals, additional restriction enzyme sites, multiple cloning sites, other coding segments, and the like, such that their overall length may vary considerably. It is therefore contemplated that a nucleic acid segment or fragment of almost any length may be employed, with the total length preferably being limited by the ease of preparation and use in the intended recombinant protocol.

For example, nucleic acid segments or fragments may be prepared that include a short contiguous stretch identical to or complementary to SEQ ID NO:1 or SEQ ID NO:3, such as about a 15, 18 or 21 nucleotide stretch, up to about 20,000, about 10,000, about 5,000 or about 3,000 base pairs in length. Nucleic acid and DNA segments with total lengths of about 1,000, about 500, about 200, about 100 and about 50 base pairs in length (including all intermediate lengths) are also contemplated to be useful.

It will be readily understood that “intermediate lengths”, in these contexts, means any length between the quoted ranges, such as 21, 22, 23, 24, 25, etc.; 30, 31, 32, etc.; 50, 51, 52, 53, etc.; 100, 101, 102, 103, etc.; 150, 151, 152, 153, etc.; including all integers through the 200-500; 500-1,000; 1,000-2,000; 2,000-3,000; 3,000-5,000; 5,000-10,000 ranges, up to and including sequences of about 12,001, 12,002, 13,001, 13,002, 15,001, 20,001 and the like.

It will also be understood that this invention is not limited to the particular nucleic acid and amino acid sequences of SEQ ID NO:1, SEQ ID NO:2, SEQ ID NO:3 and SEQ ID NO:4, respectively. Recombinant vectors and isolated DNA segments may therefore variously include the coding region from SEQ ID NO:1 or SEQ ID NO:3, coding regions bearing selected alterations or modifications in the basic coding region, or they may encode larger polypeptides that nevertheless include such coding regions or may encode biologically functional equivalent proteins or peptides that have variant amino acids sequences.

The nucleic acid and DNA segments of the present invention further include sequences that encode biologically functional equivalent Coccidioides spp. peptides that arise as a consequence of codon redundancy and functional equivalency that are known to occur naturally within nucleic acid sequences and the proteins thus encoded. Equally, functionally equivalent proteins or peptides may be created via the application of recombinant DNA technology in which changes in the protein structure may be engineered, based on considerations of the properties of the amino acids being exchanged. Changes designed by human intervention may be introduced through the application of site-directed mutagenesis techniques, e.g., to introduce improvements to the antigenicity of the protein.

III. Expression Vectors, Hosts, and Expression of Polypeptides of the Invention In Vitro and In Vivo.

The term “expression vector” refers to a polynucleotide that includes coding sequences that encode the polypeptide of the invention and provides the sequences necessary for its expression in the selected host cell. Expression vectors will generally include a transcriptional promoter and terminator, or will provide for incorporation adjacent to an endogenous promoter. Promoters that are most commonly used in recombinant DNA construction include the β-lactamase (penicillinase), lactose and tryptophan (trp) promoter systems. While these are the most commonly used, other microbial promoters have been discovered and utilized, and details concerning their nucleotide sequences have been published, enabling those of skill in the art to ligate them functionally with plasmid vectors.

The recombinant host cells of the present invention may be maintained in vitro, e.g., for recombinant protein, polypeptide or peptide production. Equally, the recombinant host cells could be host cells in vivo, such as results from immunization of an animal or human with a nucleic acid segment of the invention. Accordingly, the recombinant host cells may be prokaryotic or eukaryotic host cells, such as E. coli, Saccharomyces cerevisiae or other yeast, mammalian or human or plant host cells. It will be further appreciated by the skilled practitioner that other prokaryotic and eukaryotic cells and cell lines may be appropriate for a variety of purposes; e.g., to provide higher expression, desirable glycosylation patterns, or other features. Expression vectors will usually be plasmids, further comprising an origin of replication and one or more selectable markers. The pET28b-URE construct of the present invention is an example of such expression vectors. A YEpFLAG-1-URE construct is another example. However, expression vectors may alternatively be viral recombinants designed to infect the host, or integrating vectors designed to integrate at a preferred site within the host's genome. Examples of other expression vectors are disclosed in Molecular Cloning: A Laboratory Manual, Third Edition, Sambrook, Fritsch, and Maniatis, Cold Spring Harbor Laboratory Press, 2001.

Such polynucleotides encoding the polypeptides of the invention and expression vectors carrying the vectors can be used to produce the polypeptides in vitro or in vivo. The polypeptides so produced can be isolated according to the procedures described herein and commonly known in the art and then used in a therapeutic or immunization protocol.

One may also prepare fusion proteins and peptides, e.g., where the Coccidioides spp. peptide coding region is included within the same expression unit with other proteins or peptides having desired functions, such as for purification or immunodetection purposes (e.g., proteins that may be isolated by affinity chromatography and enzyme label coding regions, respectively), or proteins and peptides encoding additional antigens capable of eliciting an immunostimulatory response in a subject (e.g., such as the Coccidioides spp. polypeptides Ag2/Pra, Gel1, Csa, or non-Coccidioides protein antigens or toxoids, such as tetanus toxoid, diphtheria toxoid, cholera toxoid, ovalbumin, or keyhole limpet haemocyanin).

IV. How the Polypeptide May Be Isolated.

The peptides and polypeptides of the present invention, when produced, can be purified by isolation and purification methods for proteins generally known in the field of protein chemistry. Within the context of the present invention, “isolated” means separated out of its natural environment. An “isolated polypeptide” is, in this context, a substantially pure polypeptide.

The term “substantially pure polypeptide” means a polypeptide that has been separated from at least some of those components which naturally accompany it, such as other contaminating polypeptides, polynucleotides, and or other biological materials often found in cell extracts. Typically, the protein is substantially pure when it is at least 60%, by weight, free from the proteins and other naturally-occurring organic molecules with which it is naturally associated in vivo. Preferably, the purity of the preparation is at least 75%, more preferably at least 90%, and most preferably at least 99%, by weight. A substantially pure Ure polypeptide may be obtained, for example, by extraction from a natural source, or by expression of a recombinant nucleic acid encoding an immunoreactive Ure polypeptide, such as the nucleic acid molecule shown as SEQ ID NO: 3, using methods described herein. In addition, an amino acid sequence consisting of at least an immunogenic portion of the amino acid sequence of SEQ ID NO: 4 can be chemically synthesized in a substantially pure form.

Methods of purification include, for example, extraction, recrystallization, ammonium sulfate precipitation, sodium sulfate, centrifugation, dialysis, ultrafiltration, adsorption chromatography, ion exchange chromatography, hydrophobic chromatography, normal phase chromatography, reversed-phase chromatography, gel filtration method, gel permeation chromatography, affinity chromatography, electrophoresis, countercurrent distribution, combinations of these, and other know protein or peptide purification methods are well known to those of skill in the art and can be used herein.

Purity can be measured by any appropriate method, e.g., HPLC analysis, immunoaffinity chromatography using an antibody specific for the Ure polypeptide, polyacrylamide gel electrophoresis, and the like.

A Coccidioides spp. polypeptide that is “isolated to homogeneity,” as applied to the present invention, means that the Coccidioides spp. polypeptide has a level of purity where the Coccidioides spp. polypeptide is substantially free from other proteins, peptides and biological components. For example, a isolated Coccidioides spp. polypeptide will often be sufficiently free of other peptide and protein components so that sequencing may be performed successfully or that pharmaceutically acceptable formulations can be created. However, this does not exclude the re-mixing of the peptides of the invention, once isolated, with other vaccine components.

V. Preparation and Formulation of Vaccines.

The polypeptides and formulations employing the polypeptides may also be in the form of a peptide salt thereof. In view of the utility of the polypeptides of the present invention, preferred salts include those salts that are pharmaceutically acceptable for administration into a subject patient.

The polypeptides of the present invention may form a salt by addition of an acid. Examples of the acid include inorganic acids (such as hydrochloric acid, hydrobromic acid, phosphoric acid, nitric acid, and sulfuric acid) or organic carboxylic acids (such as acetic acid, propionic acid, maleic acid, succinic acid, malic acid, citric acid, tartaric acid, and salicylic acid), acidic sugars such as glucuronic acid, galacturonic acid, gluconic acid, ascorbic acid, etc., acidic polysaccharides such as hyaluronic acid, chondroitin sulfates, alginic acid, or organic sulfonic acids (such as methanesulfonic acid, and p-toluenesulfonic acid), etc.

The polypeptides of the present invention may also form a salt with a basic substance. Examples of these basic salts include, for example, salts with inorganic bases such as alkali metal salts (sodium salt, lithium salt, potassium salt, etc.), alkaline earth metal salts, ammonium salts, and the like or salts with organic bases, such as diethanolamine salts, cyclohexylamine salts, and the like.

In one embodiment of the present invention, the various polypeptides of the present invention may be admixed in various combinations and or admixed with other known proteins or peptides, which are known or believed to facilitate an immunological response, thereby providing protection against Coccidioides spp. infection. In an alternative embodiment, the polypeptides of the present invention may be administered separately, i.e., at different time points, from each or from other proteins or peptides, which are known or believed to facilitate an immunological response, thereby providing protection against Coccidioides spp. infection. For example, the peptide of amino acids 1 to 166 of SEQ ID NO:4 can be combined with one or more additional Coccidioides spp. polypeptides or antigens, such as Ag2/Pra, Csa Gel1, or non-Coccidioides protein antigens or toxoids, such as tetanus toxoid, diphtheria toxoid, cholera toxoid, ovalbumin (OVA), or keyhole limpet haemocyanin (KLH).

The pharmaceutically acceptable carriers which can be used in the present invention include, but are not limited to, an excipient, a stabilizer, a binder, a lubricant, a colorant, a disintegrant, a buffer, an isotonic agent, a preservative, an anesthetic, and the like which are commonly used in a medical field.

Also, the dosage form, such as injectable preparations (solutions, suspensions, emulsions, solids to be dissolved when used, etc.), tablets, capsules, granules, powders, liquids, liposome inclusions, ointments, gels, external powders, sprays, inhalating powders, eye drops, eye ointments, suppositories, pessaries, and the like, can be used appropriately depending on the administration method, and the polypeptides of the present invention can be accordingly formulated. Pharmaceutical formulations are generally known in the art and are described, for example, in Chapter 25.2 of Comprehensive Medicinal Chemistry, Volume 5, Editor Hansch et al, Pergamon Press 1990.

The present invention also provides compositions containing the polypeptides or fragments thereof containing one or more suitable adjuvants commonly used in the field of immunology and medicine to enhance the immune response in a subject. Examples of such adjuvants include monophosphoryl lipid A (MPL), a detoxified derivative of the lipopolysaccharide (LPS) moiety of Salmonella minnesota R595, which has retained immunostimulatory activities and has been shown to promote Th1 responses when co-administered with antigens (see U.S. Pat. No. 4,877,611; Tomai et al., Journal of Biological Response Modifiers. 1987. 6:99-107; Chen et al., Journal of Leukocyte Biology 1991. 49:416-422; Garg & Subbarao. Infection and Immunity. 1992. 60(6):2329-2336; Chase et al., Infection and Immunity. 1986. 53(3):711-712; Masihi et al, Journal of Biological Response Modifiers. 1988. 7:535-539; Fitzgerald, Vaccine 1991. 9:265-272; Bennett et al, Journal of Biological Response Modifiers 1988. 7:65-76; Kovach et al., Journal of Experimental Medicine, 1990. 172:77-84; Elliott et al., Journal of Immunology. 1991.10:69-74; Wheeler A. W., Marshall J. S., Ulrich J. T., International Archives of Allergy and Immunology October 2001;126(2):135-9; and Odean et al., Infection and Immunity 1990. 58(2):427-432); MPL derivatives (see U.S. Pat. No. 4,987,237) other general adjuvants (see U.S. Pat. No. 4,877,611); CpG and ISS oligodeoxynucleotides (see U.S. Pat. No. 6,194,388; U.S. Pat. No. 6,207,646; U.S. Pat. No. 6,239,116; U.S. Pat. No. 6,339,068; McCluskie, M. J., and H. L. Davis. Vaccine 2002. 19:413-422; Ronaghy A, Prakken B J, Takabayashi K, Firestein G S, Boyle D, Zvailfler N J, Roord S T, Albani S, Carson D A, Raz E. Immunostimulatory DNA sequences influence the course of adjuvant arthritis. Journal of Immunology 2002. 168(1):51-6.; Miconnet et al (2002) 168(3) Journal of Immunology pp 1212-1218; Li et al (2001) Vaccine 20(1-2):148-157; Davis (2000) Devopmental Biology 104:165-169; Derek T. O'Hagan, Mary Lee MacKichan, Manmohan Singh, Recent developments in adjuvants for vaccines against infectious diseases, Biomolecular Engineering 18 (3) (2001) pp. 69-85; McCluskie et al (2001) Critical Reviews in Immunology 21(1-3):103-120); trehalose dimycolate (see U.S. Pat. No. 4,579,945); amphipathic and surface active agents, e.g., saponin and derivatives such as QS21 (see U.S. Pat. No. 5,583,112); oligonucleotides (Yamamoto et al, Japanese Journal of Cancer Research, 79:866-873, 1988); detoxified endotoxins (see U.S. Pat. No. 4,866,034); detoxified endotoxins combined with other adjuvants (see U.S. Pat. No. 4,435,386); combinations with QS-21 (see U.S. Pat. No. 6,146,632); combinations of detoxified endotoxins with trehalose dimycolate and endotoxic glycolipids (see U.S. Pat. No. 4,505,899); combinations of detoxified endotoxins with cell wall skeleton (CWS) or CWS and trehalose dimycolate (see U.S. Pat. Nos. 4,436,727, 4,436,728 and 4,505,900); combinations of just CWS and trehalose dimycolate, without detoxified endotoxins (as described in U.S. Pat. No. 4,520,019); chitosan adjuvants (see U.S. Pat. Nos. 5,912,000; 5,965,144; 5,980,912; Seferian, P. G., and Martinez, M. L. Immune stimulating activity of two new chitosan containing adjuvant formulations (2001) Vaccine. 2000. 19(6):661-8). All of the references cited in this paragraph are incorporated herein by reference.

In another embodiment, the antigenic compositions of the present invention can be administered as an adsorbed vaccine or immunostimulatory composition as described in Matheis et al. (Matheis, M., Zott, A., Schwanig, M. The role of the adsorption process for production and control combined adsorbed vaccines. Vaccine. 2000. 20:67-73), which is incorporated herein by reference.

In another embodiment, various adjuvants, even those that are not commonly used in humans, may be employed in animals where, for example, one desires to subsequently obtain activated T cells or to protect valuable or valued animals from infection due to Coccidioides spp.

VI. Administration of Vaccines

As used herein the subject that would benefit from the administration of the polypeptide and or nucleotide vaccines and formulations described herein include any mammal that can benefit from protection against Coccidioides spp. infection. In a preferred embodiment, the subject is a human. In a second embodiment, the subject is a domestic animal, including but not limited to dog, cat, horse, bovine (meaning any sex or variety of cattle) or other such domestic animals.

By polypeptides capable of eliciting an immune response in a subject human, including vaccination, the invention covers any polypeptide, peptide, peptide mimic, or chemical product capable of inducing an immune reaction that results in or augments the subject's ability to mount some level of immune protection inhibiting Coccidioides spp. infection. In one embodiment, the Coccidioides spp. is Coccidioides immitis. In another embodiment, the Coccidioides spp. is Coccidioides posadasii.

As used herein, “inhibit”, “inhibiting” or “inhibition” includes any measurable or reproducible reduction in the infectivity of Coccidioides spp. in the subject patient. “Reduction in infectivity” means the ability of the subject to prevent or limit the spread of Coccidioides spp. fungus in tissues or organs exposed to or infected by said fungus. Furthermore, “amelioration”, “protection”, “prevention” and “treatment” mean any measurable or reproducible reduction, prevention, or removal of any of the symptoms associated with Coccidioides spp. infectivity, and particularly, the prevention, or amelioration of Coccidioides spp. infection and resultant pathology itself.

Optimum doses of polypeptide that elicit an inhibiting response can be determined through experimentation. Typically, one skilled in the art would design such experiments using animal models to test a range of doses that would result in both inhibitory and non-inhibitory responses, allowing for the selection of appropriate doses.

The dosages used in the present invention to provide immunostimulation include from about 0.1 μg to about 500 μg, which includes, 0.5, 1.0, 1.5, 2.0, 5.0, 10, 15, 20, 25, 30, 35, 40, 45, 50, 55, 60, 65, 70, 75, 80, 85, 90, 95, 100, 150, 200, 250, 300, 350, 400, and 450 μg, inclusive of all ranges and subranges there between. Such amount may be administered as a single dosage or may be administered according to a regimen, including subsequent booster doses, whereby it is effective; e.g., the compositions of the present invention can be administered one time or serially over the course of a period of days, weeks, months and or years.

The polypeptide compositions of the present invention can be administered by any suitable administration method including, but not limited to, injections (subcutaneous, intramuscular, intracutaneous, intravenous, intraperitoneal), eye dropping, instillation, percutaneous administration, transdermal administration, oral administration, intranasal administration, inhalation, etc.

VII. Other Uses.

Also included within the scope of the present invention are kits suitable for providing one or more of the polypeptides of the invention. For example, in such a kit one vial can comprise the polypeptides of the invention admixed with a pharmaceutically acceptable carrier, either in a aqueous, non-aqueous, or dry state; and a second vial which can carry immunostimulatory agents, and or a suitable diluent for the peptide composition, which will provide the user with the appropriate concentration of peptide to be delivered to the subject. In one embodiment, the kit will contain instructions for using the polypeptide composition and other components; as included, such instructions can be in the form of printed, electronic, visual, and or audio instructions. The vaccinations will normally be at from two to twelve week intervals, more usually from three to five week intervals. Periodic boosters at intervals of 1-5 years, usually three years, will be desirable to maintain protective levels. The course of the immunization may be followed by assays for activated T cells produced, skin-test reactivity, or other indicators of an immune response to Coccidioides spp.

The polypeptide of the invention can be used to detect the presence of antibodies in the sera of patients potentially infected with Coccidioides spp. Antibodies that react specifically with the inventive polypeptides can be used to detect the presence of circulating antigens in the sera of patients potentially infected with Coccidioides spp. Such detection systems include radioimmunoassays and various modifications thereof which are well known to those skilled in the art. In addition, the polypeptide of the invention can be used to detect the presence of a cell-mediated immune response in a biological sample. Such assay systems are also well-known to those skilled in the art and generally involve the clonal expansion of a sub-population of T cells or the production of cytokines in response to stimuli from the polypeptide or detection of reactive T cells by flow cytometry or other methods known to those skilled in the art; e.g., methods described by Richards et al. (Richards, J. O., Ampel, N. M., Galgiani, J. N. and Lake, D. F.). When so-used, the humoral and or cell-mediated response of a patient can be determined and monitored over the course of the disease. Methods of generating antibodies directed to a specific peptide fragment are known in the art. Examples of such methods are disclosed in Antibodies, A Laboratory Manual, Harlow and Lane, Cold Spring Harbor Press, 1988, herein incorporated by reference.

Having generally described this invention, a further understanding can be obtained by reference to certain specific examples that are provided herein for purposes of illustration only, and are not intended to be limiting unless otherwise specified.

### EXAMPLES

### Example 1

Immune Response and Protection Studies in Mice Immunized with Recombinant Ure Vaccines Derived from Coccidioides posadasii

Materials and Methods

Purification of recombinant proteins. The protocols for expression and purification of rURE and rHSP60 of Coccidioides spp. have been reported elsewhere, incorporated by reference in their entirety (Thomas, P. W., E. E. Wyckoff, E. J. Pishko, J. -J. Yu, T. N. Kirkland, and G. T. Cole. 1997. The hsp60 gene of the human pathogenic fungus Coccidioides immitis encodes a T-cell reactive protein. Gene 199:83-91; Yu, J. -J., S. L. Smithson, P. W. Thomas, T. N. Kirkland, and G. T. Cole. 1997. Isolation and characterization of the urease gene (URE) from the pathogenic fungus Coccidioides immitis. Gene 198:387-391). Endotoxin contamination of each stock solution of recombinant protein (1 mg/ml) solubilized in phosphate-buffered saline (PBS; 0.1 M, pH 7.4) was assayed using a Limulus amebocyte lysate kit (QCL-1000; BioWhittaker, Walkersville, Md.). All preparations had fewer than 30 endotoxin units (150 ng of endotoxin) per μg of protein.

CpG DNA. Unmethylated CpG dinucleotides present in a synthetic oligodeoxynucleotide (ODN) preparation (CpG ODN; Integrated DNA Technologies, Inc., Coralville, Iowa) were used as an immunoadjuvant in this study as previously described (Hung, C. -Y., N. M. Ampel, L. Christian, K. R. Seshan, and G. T. Cole. 2000. A major cell surface antigen of Coccidioides immitis which elicits both humoral and cellular immune responses. Infection and Immunity 68:584-593). The CpG ODN sequence used to immunize mice (SEQ ID NO:5-TCCATGACGTTCCTGACGTT [CpG motifs are underlined]) was the same as that reported by Chu et al. (Chu, R. S., O. S. Targoni, A. M. Krieg, P. V. Lehmann, and C. V. Harding. 1997. CpG oligodeoxynucleotides act as adjuvants that switch on T helper 1 (Th 1) immunity. Journal of Experimental Medicine 186:1623-1631) (ODN 1826). The ODN was phosphorothioate modified to increase resistance to nuclease degradation. The CpG ODN preparation was dissolved in PBS (1 mg/ml) and used as a stock solution for subsequent immunizations.

Animals. All protection experiments were conducted with 10-week-old, female BALB/c mice supplied by the National Cancer Institute (Bethesda, Md.). BALB/c mice have been shown to be highly susceptible to C. immitis infection (Kirkland, T. N., and J. Fierer. 1983. Inbred mouse strains differ in resistance to lethal Coccidioides immitis infection. Infection and Immunity 40:912-916).

ELISA. The indirect enzyme-linked immunosorbent assay (ELISA) was performed for examination of Ig titers to selected antigens in immunized and C. posadasii-infected mice as previously described (Abuodeh, R. O., L. F. Shubitz, E. Siegel, S. Snyder, T. Peng, K. I. Orsborn, E. Brummer, D. A. Stevens, and J. N. Galgiani. 1999. Resistance to Coccidioides immitis in mice after immunization with recombinant protein or a DNA vaccine of a proline-rich antigen. Infection and Immunity 67:2935-2940). Heat-inactivated sera from mice immunized with rURE, rHSP60, or BSA plus CpG ODN as described above were tested for reactivity with the respective, purified antigens. Sera were collected separately from three mice in each group by heart puncture exsanguination at 12 days after intraperitoneal (i.p.) challenge with C. posadasii as described below. Control sera were obtained from three nonimmunized, infected mice. The final concentration of each purified recombinant antigen and BSA applied to the wells of the microtiter plates (Falcon no. 3077; Becton Dickinson) was 10 ng in 100 μl of PBS. The plates were washed with PBS which contained 0.33% (vol/vol) Brij 35 detergent (Sigma) and blocked with the same buffer plus 5% (vol/vol) FCS (Sigma). A fourfold serial dilution (1:10 to 1:10,240) of each serum sample in PBS was used to test binding of Igs (total Ig, IgG2a, and IgG1) to the purified antigens as previously described (Abuodeh, et al. 1999). After incubation of the sera with the antigens in the microtiter plates (24° C., 2 h), the wells were washed with PBS that contained Brij 35 as above. Alkaline phosphatase conjugated with goat anti-mouse Ig (IgM, IgG, and IgA; heavy and light chains), goat anti-mouse IgG2a, or goat anti-mouse IgG1 (Southern Biotechnology Associates, Inc., Birmingham, Ala.) was added to the wells at a dilution of 1:1,000 in PBS to detect bound, primary antibody. The plates were incubated at room temperature for 15 min, and the reaction was stopped by addition of 1.0 NH₂SO₄. Antibody adsorption was determined by OD at 405 nm. Nonspecific antibody adsorption to each antigen was determined by OD after incubation of the nonimmune, control serum samples diluted 1/2,000 in antigen-coated wells of the microtiter plates as above. The antibody titer of each serum sample from the immunized, infected mice is defined as the dilution that yielded an OD in the ELISA that was 50% of the maximum OD for each antigen after subtraction of the value for nonspecific antibody adsorption. The data were determined as mean values for the three test sera in each group ±standard error of the mean.

Protection assays. Results of protection assays are based on infection with a single strain (C735) of C. posadasii (previously known as an isolate of C. immitis), which has been previously shown to be highly virulent in BALB/c mice (Cole, G. T., and T. N. Kirkland. 1991. Conidia of Coccidioides immitis: their significance in disease initiation, p. 403-443. In G. T. Cole, and H. C. Hoch (ed.), The fungal spore and disease initiation in plants and animals. Plenum Press, New York, N.Y. ). C. posadasii strain C735 was grown on GYE plates at 30° C. for 6 to 8 weeks, and arthroconidia were then harvested by passing a suspension of the cells in sterile saline through an autoclaved glass wool column to remove hyphal fragments. The concentration of the stock suspension of arthroconidia was adjusted to 10³ viable cells/ml of saline. BALB/c mice were immunized with selected antigens using the protocol described above. The animals were then challenged by the i.p. route at 14 days after the last protein immunization. The inoculum contained 100 viable arthroconidia in 100 μl of PBS. As previously argued (Kirkland, T. N., P. W. Thomas, F. Finley, and G. T. Cole. 1998. Immunogenicity of a 48-kilodalton recombinant T-cell-reactive protein of Coccidioides posadasii. Infection and Immunity 66:424-431), the i.p. route of inoculation permitted more precise control of the size of the inoculum which was actually delivered to host tissues and better reproducibility of levels of coccidioidal infection in the lungs than the intranasal route of challenge. The i.p. route of challenge has been used to evaluate other cloned antigens of C. posadasii as potential vaccine candidates (Abuodeh, et al. 1999; Jiang, C., D. M. Magee, T. N. Quitugua, and R. A. Cox. 1999. Genetic vaccination against Coccidioides immitis: comparison of vaccine efficacy of recombinant antigen 2 and antigen 2 cDNA. Infection and Immunity 67:630-635; Kirkland, T. N., F. Finley, K. I. Orsborn, and J. N. Galgiani. 1998. Evaluation of the proline-rich antigen of Coccidioides posadasii as a vaccine candidate in mice. Infection and Immunity 66:3519-3522, Kirkland, T. N., P. W. Thomas, F. Finley, and G. T. Cole. 1998. Immunogenicity of a 48-kilodalton recombinant T-cell-reactive protein of Coccidioides immitis, Infection and Immunity 66:424-431). Protection was evaluated both by residual C. posadasii burden at 12 days postinfection and by the ability of immunized mice to survive to at least 40 days after challenge. For fungal burden evaluations, the lung and spleen homogenates of surviving mice were subjected to quantitative analyses of CFU in dilution plate cultures as previously described (Kirkland, T. N., P. W. Thomas, F. Finley, and G. T. Cole. 1998. Immunogenicity of a 48-kilodalton recombinant T-cell-reactive protein of Coccidioides immitis, Infection and Immunity 66:424-431). For both fungal burden and survival studies, each group of test and control animals consisted of at least 12 mice.

Statistical analyses. The numbers of CFU per organ were expressed on a log scale. Because these values did not fall into a normal distribution, the Mann-Whitney U test was used to compare medians in all cases. Survival differences between groups of mice were calculated for statistical significance by the Kaplan-Meier method. All statistical analyses were performed using the SPSS version 9.0 statistical package for Windows (SPSS Inc., Chicago, Ill.).

Results

Fungal burden in recombinant protein-immunized mice. Four groups of mice (12 per group) were immunized with BSA or recombinant protein, challenged with 100 arthroconidia of C. posadasii by the i.p. route, and sacrificed 12 days later to compare the number of residual organisms present in lungs and spleens. The results of a vaccination experiment showed that the rURE-immune mice had significantly lower counts of C. posadasii in both lungs and spleens compared to control mice immunized with BSA plus CpG ODN. The Mann-Whitney U test for statistical significance of the difference between CFU in organs of these two groups of mice yielded P values of 0.0001 and 0.002. The numbers of organisms present in lungs and spleens of rHSP60-immune mice did not show a statistically significant difference compared to CFU in lungs and spleens of control animals immunized with BSA plus CpG ODN (P=0.268 and 0.690, respectively.) The median number of organisms in lung homogenates of rURE-immune mice was 1.3 (log₁₀) CFU (range, 0.1 to 5.3) compared to rHSP60-immune mice, with a median of 5.5 (log₁₀) CFU (range, 0.9 to 6.2). This difference in CFU between rURE- and rHSP60-immune mice was statistically significant (P=0.023). A similar difference was seen in the spleen. The median CFU in rURE-immune mice was 1.5 (log₁₀) (range, 1.4 to 5.8), compared to the median CFU in rHSP60-immune mice of 5.2 (log₁₀) (range, 4.9 to 6.2). The difference between CFU in the spleens of rURE- and rHSP60-immune mice was also statistically significant (P=0.013). The difference between the median CFU and range of counts of organisms in lungs and spleens of control mice immunized with BSA alone compared to BSA plus CpG ODN was not statistically significant. Similar data were obtained in a second vaccination experiment with rURE compared to BSA plus CpG ODN immunization of BALB/c mice. The median CFU in the control mice was 5.0 (range, 3.0 to 6.2) in the lungs and 5.5 (range, 4.5 to 6.3) in the spleen. The medians (and ranges) of CFU in the rURE-immune mice were 0.0 (0.0 to 5.5) and 0.0 (0.0 to 6.0), respectively. The P values based on the difference between these counts in immunized versus control mice were 0.008 and 0.007, respectively, which are statistically significant.

Survival of mice immunized with recombinant protein. Mice immunized with BSA or recombinant protein plus CpG ODN and challenged by the i.p route as above (12 mice per group) were scored for survival over a 40-day period post-challenge. The results of a representative survival experiment demonstrated that control mice immunized with BSA plus immunoadjuvant typically began to die as a result of C. posadasii infection at about 12 days post-challenge, and the number of survivors sharply decreased over the following 10 days. Both rHSP60- and rURE-immune mice showed prolonged survival after i.p. infection. However, the rate of mortality among the rHSP60-immune mice during the 8-day period after the first fatality (i.e., days 16 to 23 post-challenge) was more comparable to that of the control mice than that of the rURE-immune mice. At 40 days post-challenge, only 15% of the mice immunized with rHSP60 survived, compared to 42% of the rURE-immune mice. The difference between the percentage of rURE-immune mice which survived over the 40-day period compared to control mice immunized with BSA plus CpG ODN was highly significant as determined by the Kaplan-Meier test (P=0.0002). The difference in survival between rURE- and rHSP60-immunized mice was also statistically significant (P=0.013). The results of a repeated survival experiment in which the same immunization and challenge protocols were used revealed that the percentage of rURE-immune survivors compared to that of control mice was highly significant (P<0.0001). A range of doses of rURE and rHSP60 (5, 15, and 30 μg) was tested in these survival experiments. Immunization with 5 μg of rURE using the protocol described in Materials and Methods resulted in survival of only 25% of the mice at 40 days after i.p. challenge. Immunization with 30 or 60 μg of rURE resulted in 44 or 47% survival, respectively. Immunization of BALB/c mice with 30 or 60 μg of rHSP60 resulted in survival of 12 or 16%, respectively, of the animals at 40 days postchallenge. These additional survival studies using doses of 30 and 60 μg of immunogen supported our conclusion that rURE was a better vaccine candidate than rHSP60. Recombinant protein-immunized mice which survived to 42 days post-challenge were sacrificed, and their lungs and spleens were excised, homogenized, and dilution plated to determine whether C. posadasii was present. Both lungs and spleens of rURE- and rHSP60-immune mice were positive for residual Coccidioides spp. colonies in the survival experiments. Despite the lack of complete clearance of the pathogen in the survival experiments, recombinant urease was identified as a potential vaccine candidate based on results of its evaluation in fungal burden assays and survival experiments.

Obviously, numerous modifications and variations on the present invention are possible in light of the above teachings. It is therefore to be understood that within the scope of the appended claims, the invention may be practiced otherwise than as specifically described herein.

What is claimed is:

1. A method of eliciting an immune response in a mammal, comprising introducing into the mammal, in an amount sufficient to elicit an immune response, an isolated polypeptide selected from the group consisting of: a polypeptide encoded by the nucleotide sequence of SEQ ID NO:1, a polypeptide comprised of the amino acid sequence of SEQ ID NO:2, a polypeptide encoded by the nucleotide sequence of nucleotides 100 to 1749 of SEQ ID NO:3, a polypeptide comprised of the amino acid sequence of amino acids 34 to 582 of SEQ ID NO:4, and a polypeptide comprised of the amino acid sequence of amino acids 34 to 582 of SEQ ID NO:4 with conservative amino acid substitutions.

2. The method of claim 2, wherein said mammal is a human.

3. The method of claim 2, wherein said mammal is a domestic animal selected from the group consisting of dog, cat, horse, and bovine.

4. The method of claim 1, further comprising administering one or more adjuvants.

5. The method of claim 4, wherein the one or more adjuvants are administered simultaneously.

6. The method of claim 1, further comprising a second Coccidioides spp. protein, polypeptide, or peptide.

7. A method of vaccinating a mammal from coccidioidomycosis, comprising administering into the mammal, in an amount sufficient to elicit an immune response wherein said immune response is sufficient to suppress or attenuate growth of Coccidioides spp. fungus in a mammal infected with said fungus, an isolated polypeptide selected from the group consisting of: a polypeptide encoded by the nucleotide sequence of SEQ ID NO:1, a polypeptide comprised of the amino acid sequence of SEQ ID NO:2, a polypeptide encoded by the nucleotide sequence of nucleotides 100 to 1749 of SEQ ID NO:3, a polypeptide comprised of the amino acid sequence of amino acids 34 to 582 of SEQ ID NO:4, and a polypeptide comprised of the amino acid sequence of amino acids 34 to 582 of SEQ ID NO:4 with conservative amino acid substitutions.

8. The method of claim 7 wherein said mammal is a human.

9. The method of claim 7 wherein said mammal is a domestic animal selected from the group consisting of dog, cat, horse, and bovine.

10. The method of claim 7 wherein one or more pharmaceutically acceptable carriers are administered simultaneously.

11. The method of claim 7, wherein one or more adjuvants are administered simultaneously.

12. The method of claim 7, further comprising a second Coccidioides spp. protein, polypeptide, or peptide.

13. An isolated nucleic acid selected from the group consisting of: a nucleic acid comprising the nucleotide sequence of SEQ ID NO:1, a nucleic acid encoding the polypeptide comprising the amino acid sequence of SEQ ID NO:2, a nucleic acid comprising the sequence of nucleotides 100 to 1749 of SEQ ID NO:3, a nucleic acid encoding the polypeptide comprised of amino acids 34 to 582 of SEQ ID NO:4, and a nucleic acid encoding the polypeptide comprised of amino acids 34 to 582 of SEQ ID NO:4 with conservative amino acid substitutions.

14. An expression vector comprising the nucleic acid of claim 13.

15. The expression vector of claim 14, further comprising a recombinant regulatory sequence operably linked to said nucleic acid.

16. The expression vector of claim 15, wherein said regulatory sequence is a promoter.

17. The expression vector of claim 15, wherein said regulatory sequence comprises one or more transcriptional regulatory elements that control expression of the nucleic acid in a host cell.

18. The expression vector of claim 17, wherein said host cell is selected from the group consisting of yeast, plant, animal, human and bacterial cells.

19. A host cell comprising the expression vector of claim 14.

20. The host cell of claim 19, wherein said host cell is selected from the group consisting of yeast, plant, animal, human and bacterial cells.

21. An isolated polypeptide selected from the group consisting of: a polypeptide encoded by the nucleotide sequence of SEQ ID NO:1, a polypeptide comprised of the amino acid sequence of SEQ ID NO:2, a polypeptide encoded by the nucleotide sequence of nucleotides 100 to 1749 of SEQ ID NO:3, a polypeptide comprised of the amino acid sequence of amino acids 34 to 582 of SEQ ID NO:4, and a polypeptide comprised of the amino acid sequence of amino acids 34 to 582 of SEQ ID NO:2 with conservative amino acid substitutions.

22. A composition comprising the polypeptide of claim 21.

23. The composition of claim 22, wherein said composition includes a pharmaceutically acceptable carrier.

24. The composition of claim 22, further including an adjuvant.

25. The composition of claim 22, further comprising at least a second Coccidioides spp. polypeptide.

26. A kit, comprising the isolated polypeptide of claim 21.

27. The kit of claim 26, further including an adjuvant.

28. The kit of claim 26, further including instructions for use.

29. A method of generating antibodies specific for Ure, comprising introducing into a mammal the isolated polypeptide comprised of the amino acid sequence of amino acids 34 to 582 of SEQ ID NO:4, in an amount sufficient to elicit an antibody response.

30. The method of claim 29, wherein one or more pharmaceutically acceptable carriers are administered simultaneously.

31. The method of claim 30, wherein one or more adjuvants are administered simultaneously.
